(19) United States
(12) Patent Application Publication
(10) Pub. No.: US 2026/0215973 A1
(43) Pub. Date: Jul. 30, 2026

(54) REUSABLE UNDERWEAR WITH ALTERNATIVE SEWN CONSTRUCTION THAT RESISTS LEAKS

(71) Applicant: Kimberly-Clark Worldwide, Inc., Neenah, WI (US)

(72) Inventors: Oxana Radzdolski, Franklin Lakes, NJ (US); Courtney Newman, Beacon, NY (US); Marcille F. Ruman, Oshkosh, WI (US); Palandage Nayana Deduruks Saman Kumari, Colombo (LK); Deeyayawaththe Gedara Rumesh Mahela Kandegedara, Polgahawela (LK); Dona Senita Vihangi Ashesha Hettiarachchi, Colombo (LK); Hewage Sameera Sunil Chamara, Gampaha (LK); Kusumawathi Rajakaruna, Moratuwa (LK); Thushara Nishendra Embuldeniya, Pannipitiya (LK)

(21) Appl. No.: 19/159,104

(22) PCT Filed: Feb. 28, 2024
(86) PCT No.: PCT/US2024/017726

§ 371 Date: Aug. 22, 2025

Related U.S. Application Data
(60) Provisional application No. 63/487,402, filed on Feb. 28, 2023.

(51) Int. Cl. A61F 13/49; A41B 9/12; A61F 13/15; A61F 13/42; A61F 13/475; A61F 13/494; A61F 13/496; A61F 13/513; A61F 13/537

(52) U.S. Cl. CPC A61F 13/49006; A41B 9/12; A61F 13/15268; A61F 13/42; A61F 13/4755; A61F 13/49426; A61F 13/496; A61F 13/51394; A61F 13/53743

(57) ABSTRACT

A reusable garment includes a functional layer, a barrier layer, a barrier strip, and a body fabric layer. The barrier strip may be coupled to the barrier layer and extend around an external side of a first side edge of the barrier layer such that the barrier strip defines at least a portion of an outermost side edge of the reusable garment. In some cases, the body fabric layer may be coupled to the barrier strip on a garment side of the functional layer.

### REFERENCE TO RELATED APPLICATION

This application claims the benefit of U.S. Provisional Patent Application No. 63/487,402, filed on Feb. 28, 2023, and entitled REUSABLE UNDERWEAR WITH ALTERNATIVE SEWN CONSTRUCTION THAT RESISTS LEAKS, the content of which is hereby incorporated by reference in its entirety.

### FIELD OF THE INVENTION

The present disclosure relates generally to absorbent articles intended for personal wear, and more particularly to reusable absorbent articles improved constructions that resist leaks.

### BACKGROUND

Many absorbent articles intended for personal wear, such as diapers, training pants, feminine hygiene products, adult incontinence products, bandages, medical garments, and the like are designed to be sufficiently absorbent to absorb moisture from liquid body exudates including urine, menses, blood, etc., away from the wearer to reduce skin irritation caused by prolonged wetness exposure. Reusable underwear and other similar garments are typically constructed like regular underwear (or other garment), but with added layers for absorbent and barrier properties. While such reusable garments include a barrier layer, they are not leakproof, and leakage often occurs via holes formed by stitching (and often into a body fabric) and/or via wicking across the material components. Leakage through stitching holes is defined by the transfer of liquid through the thread/stitching holes that join the gusset and main body layers together. This stitching punctures the barrier layer. Leakage due to wicking across material components is defined as wicking from the absorbent gusset materials to the inner leg bands to outer leg bands and to outer main body material. Some reusable garments have offered a sealed seam as an alternative to sewn seams. While the traditional sealed seam approach eliminates the stitching holes and leakage through stitching holes, the sealed seams do not prevent leakage due to wicking across the material components. Stitched seams suffer from similar leakage due to wicking. Moreover, sealed seams are formed using adhesives and bonding, which lock up the stretch of leg elastics, thereby compromising fit and comfort of the reusable garment.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, a reusable garment includes a functional layer, a barrier layer, a barrier strip, and a body fabric layer. The functional layer includes a body side surface on a body side of the reusable garment. The barrier strip is coupled to the barrier layer and extends around an external side of a first side edge of the barrier layer such that the barrier strip defines at least a portion of an outermost side edge of the reusable garment.

According to some embodiments, a reusable garment includes a functional layer, a barrier layer, a barrier strip, and a body fabric layer. The functional layer includes a body side surface on a body side of the reusable garment. The barrier strip is coupled to the barrier layer, and the body fabric layer is coupled to the barrier strip on a garment side of the functional layer.

According to various embodiments, a reusable garment includes a functional layer, a barrier layer, a barrier strip, and a body fabric layer. The barrier layer may include a folded barrier layer portion under a topmost surface of the barrier layer. The barrier strip may include a folded barrier strip portion folded under a topmost surface of the barrier strip. In certain embodiments, the folded barrier strip portion is disposed on a garment-facing side of the folded barrier layer portion of the barrier layer. In some embodiments, a stitch couples the folded barrier layer portion to the folded barrier strip portion.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 is a front outer view of a reusable garment with a gusset according to embodiments.

FIG. 2 is a front inner view of the reusable garment of FIG. 1.

FIG. 3 is a top view of the gusset of FIG. 1.

FIG. 4 is a sectional view of the gusset of FIG. 1 taken at a leg side of the gusset and along line 4-4 in FIG. 3.

FIG. 5 is a sectional view of the gusset of FIG. 1 taken at a front side of the gusset along line 5-5 in FIG. 3.

FIG. 6 is a sectional view of a gusset for a reusable garment according to embodiments.

FIG. 7 is a sectional view of a gusset for a reusable garment according to embodiments.

FIG. 8 is a sectional view of a gusset for a reusable garment according to embodiments.

FIG. 9 is a sectional view of a gusset for a reusable garment according to embodiments.

FIG. 10 is a sectional view of a gusset for a reusable garment according to embodiments.

FIG. 11 is an inner view of a reusable garment with a gusset according to embodiments.

FIG. 12 is an inner view of a reusable garment with a gusset according to embodiments.

FIG. 13 is an outer view of the reusable garment of FIG. 12.

FIG. 14 is a top view of the gusset of FIG. 12.

FIG. 15 is a sectional view of a gusset for a reusable garment according to embodiments.

FIG. 16 is a sectional view of a gusset for a reusable garment according to embodiments.

FIG. 17 is a sectional view of a gusset for a reusable garment according to embodiments.

FIG. 18 is another sectional view of the gusset of FIG. 17 illustrating a change in height in a barrier strip according to embodiments.

FIG. 19 is a top view of a gusset for a reusable garment according to embodiments.

FIG. 20 is a sectional view of the gusset of FIG. 18 taken along line 20-20 in FIG. 19.

FIG. 21 is a sectional view of the gusset of FIG. 18 taken along line 21-21 in FIG. 19.

FIG. 22 is a sectional view of a gusset taken at a front side of the gusset.

### DETAILED DESCRIPTION

Described herein are improved gussets for reusable garments as well as reusable garments incorporating such gussets. In certain embodiments, the gussets described herein may provide an improved barrier that minimizes and/or prevents leakage compared to traditional approaches. In some embodiments, the gussets described herein may provide an improved barrier at side edges that minimizes and/or prevents leakage from material wicking. In various embodiments, the gussets described herein may eliminate stitching holes that extend through all layers of the gusset, thereby preventing leakage through such stitching holes. In some embodiments, the gussets described herein may provide improved leakage control while maintaining leg elastic function and thereby maintaining comfort and fit of the reusable garment to a wearer and/or openings of the gusset. Various other benefits and advantages may be realized with the systems and methods provided herein, and the aforementioned advantages should not be considered limiting.

FIGS. 1-5 illustrate an example of a reusable garment 100 with a gusset 102 according to embodiments. In FIGS. 1-5, the reusable garment 100 is illustrated as reusable underwear. However, the particular reusable underwear illustrated should not be considered limiting, and in further embodiments, other types of reusable underwear and/or other types of reusable garments 100 may be utilized with the gusset 102 as desired, such as but not limited to a brief, an undergarment, a bathing suit, a pajama, a shorts, a leotard, a legging, a pants, a skort, a panty, a boy short, a bikini, a thong, an overalls, a bodysuit, a leotard, a tights, a pantyhose, a boxers, a biking shorts, and/or a compression shorts.

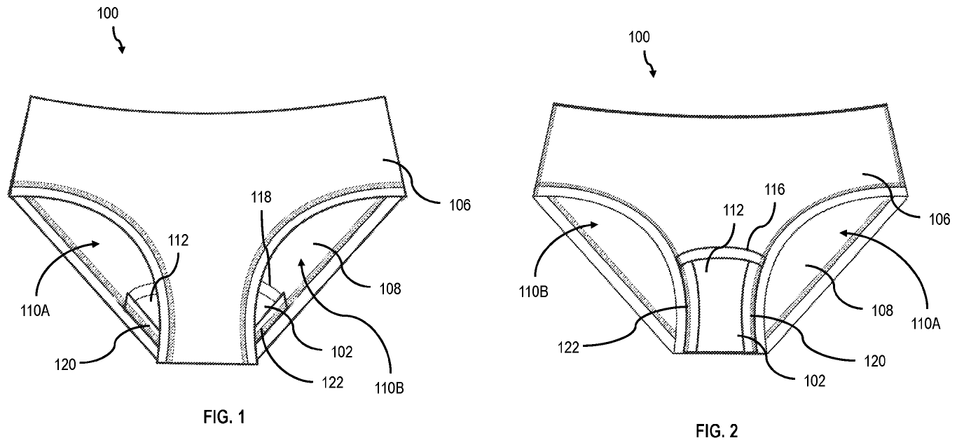

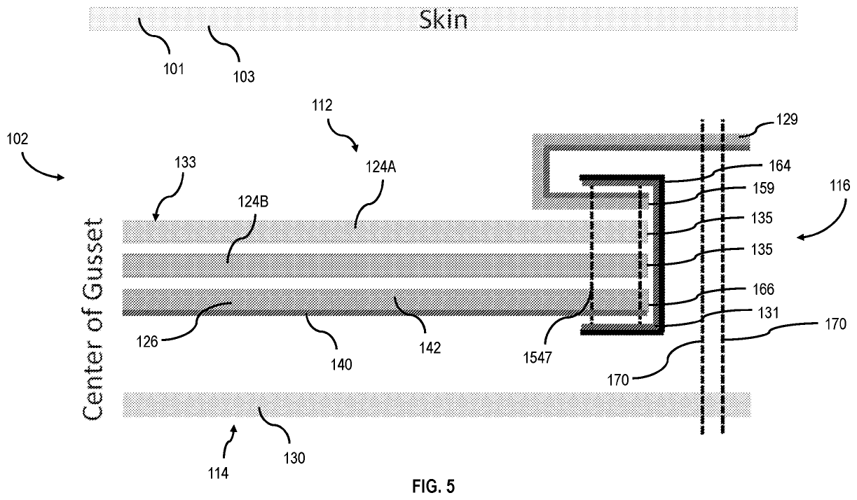

As illustrated in FIGS. 1 and 2, the reusable garment 100 may include a front portion 106 and a rear portion 108, and the gusset 102 may be provided between the front portion 106 and the rear portion 108. In some embodiments, the gusset 102 may be coupled to the front portion 106 and/or the rear portion 108 via stitching and/or other suitable techniques as discussed below with reference to FIG. 5. Leg openings 110A-B are defined by the reusable garment 100.

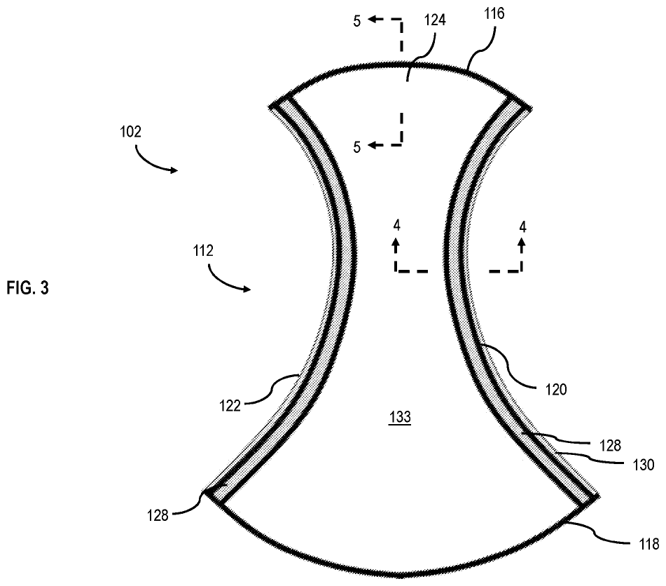

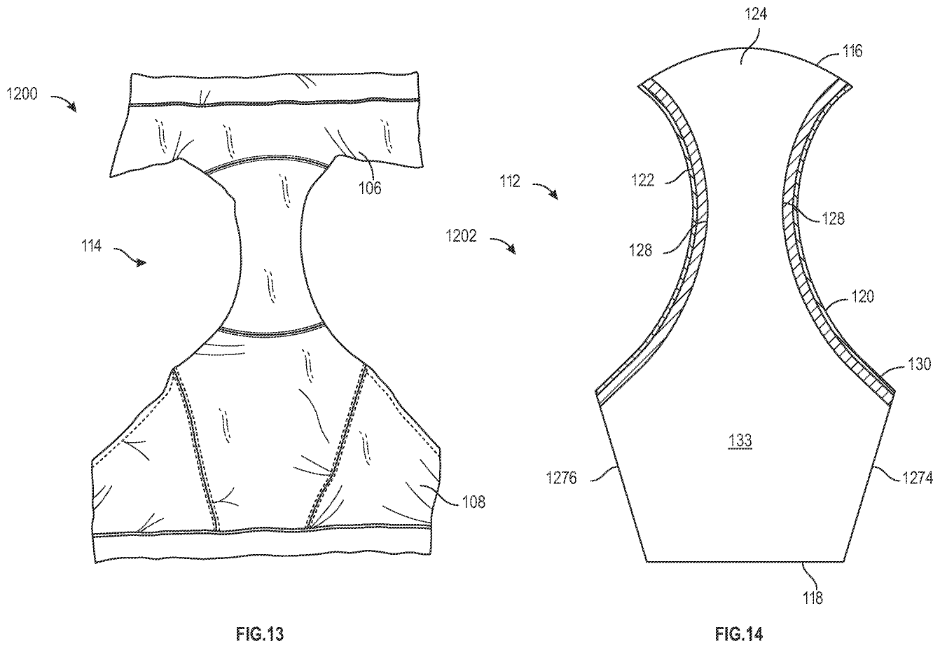

Referring to FIGS. 3-5, the gusset 102 generally includes a top side (or body side) 112 that faces a user 101 when worn a bottom side (or garment side) 114 that faces away from the user 101 when worn. The gusset 102 also includes a front side 116, a back side 118, a first side 120, and a second side 122. In certain embodiments, one or more of the sides 116, 118, 120, 122 may be curved or non-linear as illustrated in FIGS. 1-5, although in other embodiments, the one or more sides 116, 118, 120, 122 may include one or more linear portions. As such, the gusset 102 may have various shapes or profiles as desired, and the shape of the gusset 102 illustrated in FIG. 3 should not be considered limiting. As a non-limiting example, FIGS. 12-14 illustrate a gusset with another shape or profile different from that illustrated in FIG. 3 and includes linear portions 1274, 1276. In certain embodiments, the shape of the gusset 102 may at least partially depend on a type of reusable garment incorporating the gusset 102.

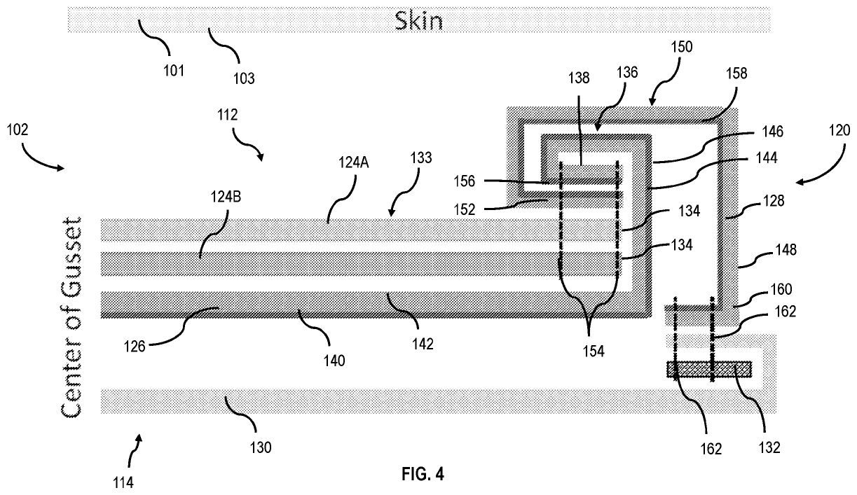

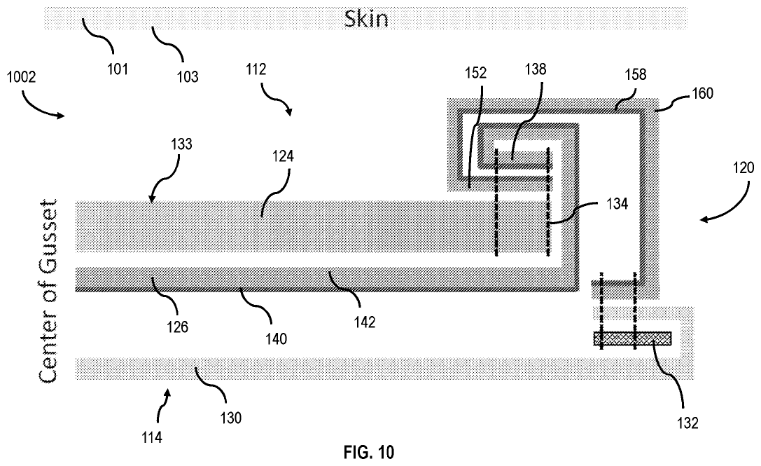

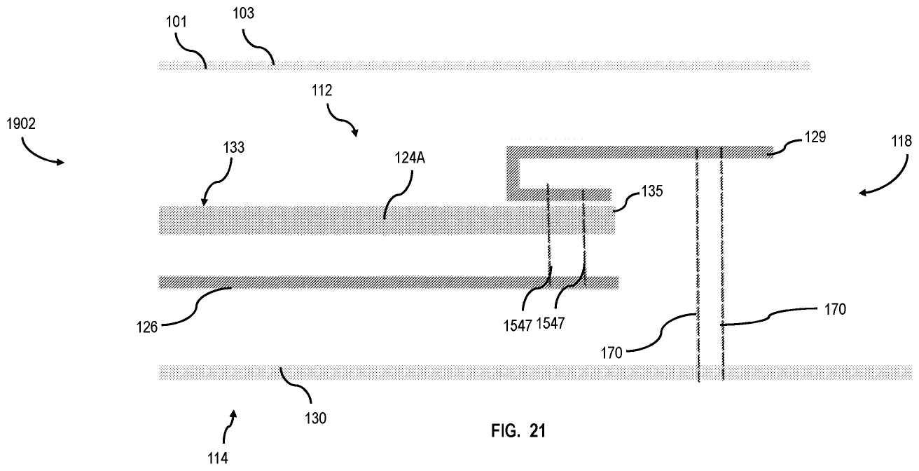

Referring to FIGS. 4 and 5, the gusset 102 generally includes one or more functional layers 124, a barrier layer 126, a barrier strip 128, and a body fabric layer 130. In the embodiment illustrated in FIGS. 4 and 5, the one or more functional layers 124 include two functional layers 124A-B. However, the number of functional layers 124 should not be considered limiting, and in other embodiments, the gusset 102 may include a single functional layer 124 (see, e.g., FIGS. 10 and 21) and/or more than two functional layers 124 as desired.

In some embodiments, an arrangement or construction of layers of the gusset 102 at the first side 120 and/or the second side 122 may be different from an arrangement or construction of layers of the gusset 120 at the front side 116 and/or the back side 118. FIGS. 4 and 5 illustrate non-limiting examples of the arrangement or construction of layers of the gusset 102 at the first side 120 and/or the second side 122 different from an arrangement or construction of layers of the gusset 120 at the front side 116 and/or the back side 118. In other embodiments, an arrangement or construction of layers of the gusset 102 at the first side 120 and/or the second side 122 may be the same as an arrangement or construction of layers of the gusset 120 at the front side 116 and/or the back side 118. As a non-limiting example, the sides 116, 118, 120, 122 may have a construction similar to that illustrated in FIG. 4. As a further non-limiting example, the sides 116, 118, 120, 122 may each have at least (i) the barrier layer 126 at least on a body side of the gusset 102 and (ii) the barrier strip 128 with a free-floating topmost surface 150 (discussed in detail below). In other embodiments, other constructions may be implemented as desired.

The one or more functional layers 124 may be various absorbent materials and/or moisture-wicking materials as desired. As non-limiting examples, the one or more functional layers 124 may be a material including, but not limited to, fabric, natural yarn, synthetic yarn, synthetic fabric, nonwovens, wovens, microfiber terry fabrics, super absorbent fabrics, cellulose, spacer fabrics, polyurethane, wool, polypropylene, polyamide, polyester, cotton, silk, elastane, rubber, plastic, metal, merino wool, rayon, linen, bamboo, taffeta, Gore-Tex®, X-static®, Tencel™, lyocell, modal, combinations thereof, or other suitable materials as desired. In certain embodiments, a single functional layer 124 may be constructed via various techniques as desired such that the functional layer 124 provides both moisture-wicking and absorbent properties. As a non-limiting example, the functional layer 124A may be constructed from a material or combination of materials such that the functional layer 124A provides both moisture-wicking and liquid absorption. In other embodiments, the moisture-wicking material and the absorbent material may be provided as discrete layers. As non-limiting examples and referring to FIGS. 4 and 5, the functional layer 124A may be a moisture-wicking layer and the functional layer 124B may be an absorbent layer, or, alternatively, the functional layer 124A may be an absorbent layer and the functional layer 124B may be a moisture-wicking layer.

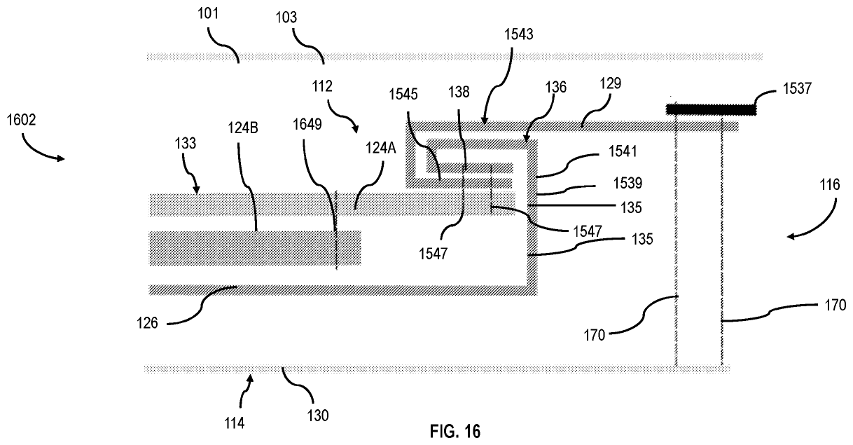

In some embodiments with a plurality of functional layers 124, the functional layers 124 may have similar shapes, sizes, and/or profiles. In other embodiments with a plurality of functional layers, the functional layers 124 may have different or non-homogeneous shapes, sizes, and/or profiles. Stated differently, the shape, size, and/or profile of one functional layer need not be the same as the shape, size, and/or profile of another functional layer. FIGS. 16-18, 20, and 21 illustrate non-limiting examples where the shape, size, and/or profile of functional layers 124 are dissimilar. As a further non-limiting example, a thickness of one functional layer 124 may be the same as or different from a thickness of another functional layer 124. Additionally, or alternatively, in embodiments with a plurality of functional layers 124, the edges of the functional layers 124 optionally may be aligned along at least a portion of one or more of the sides 116, 118, 120, 122 (see, e.g., FIGS. 4-9, 15, and 20). However, the edges of the functional layers 124 need not be aligned (partially or completely), and in some embodiments, the edge of one functional layer 124 may be offset from the edge of another function layer 124 at one or more of the sides 116, 118, 120, 122 (see, e.g., FIGS. 16-18).

The barrier layer 126 may be constructed from various materials such that the barrier layer 126 is substantially liquid-impervious to thereby prevent moisture from penetrating through the gusset 102 from the one or more functional layers 124 and/or out the side edge(s) 134. Suitable materials include, but are not limited to, waterproof materials, water resistant materials, coated materials, laminated materials, nonwovens, films, combinations thereof, and/or other barrier technologies as desired. Such materials may be provided in one or more layers to form the barrier layer 126. In the embodiment illustrated in FIGS. 4 and 5, the barrier layer 126 is a laminate having a barrier side 140 and a fabric side 142. In this embodiment, the fabric side 142 may be a material such as but not limited to a fabric, natural yarn, synthetic yarn, synthetic fabric, polyurethane, wool, polypropylene, polyamide, polyester, cotton, silk, elastane, rubber, plastic, metal, merino wool, rayon, linen, bamboo, taffeta, Gore-Tex®, X-static®, Tencel™, lyocell, modal, combinations thereof, or other suitable materials as desired. The barrier side 140 may be a film or coating, such as but not limited to a polyurethane coating and/or other suitable material as desired. In these embodiments, the barrier side 140 and the fabric side 142 may be provided in various orientations or configurations relative to the one or more functional layers 124. Optionally, the barrier layer 126 may be constructed from a material that is treated to be hydrophobic and/or liquid repellant. Optionally, the material of the barrier layer 126 may be treated to be stain-resistant.

Similar to the barrier layer 126, the barrier strip 128 may be constructed from various materials such that the barrier strip 128 is substantially liquid-impervious (e.g., natively or due to treatment). In some embodiments, the barrier strip 128 may include the same materials and/or similar construction as the barrier layer 126, although it need not in other embodiments. As non-limiting examples, the barrier strip 128 may be waterproof materials, water resistant materials, coated materials, laminated materials, nonwovens, films, combinations thereof, and/or other barrier technologies as desired, and such materials may be provided in one or more layers to form the barrier strip 128. In the embodiment illustrated in FIGS. 4 and 5, the barrier strip 128 is a laminate having a barrier side 158 and a fabric side 160. In this embodiment, the fabric side 160 may be a material similar to those suitable for the fabric side 142 of the barrier layer 126, and the barrier side 158 may be a material similar to those suitable for the barrier side 140 to the barrier layer 126.

The body fabric layer 130 may include any suitable material for use in a garment, including but not limited to fabric, natural yarn, synthetic yarn, synthetic fabric, polyurethane, wool, polypropylene, polyamide, polyester, combinations thereof, and/or other suitable materials as desired. Optionally, the body fabric layer 130 (and/or leg band coverings) may also have hydrophobic and/or liquid repellent properties. As a non-limiting example, the body fabric layer 130 may be treated with a fabric softener or other liquid repellent. Various other materials may be used as the body fabric layer 130 as desired, and the aforementioned examples should not be considered limiting.

Optionally, the gusset 102 may include leg elastic 132, although it need not in other embodiments. As used herein, “elastic,” “elastomeric,” and the like refer to the ability of a material or composite to be elongated by at least about 50 percent and upon relaxation to return to within at least 50 percent of its original length.

FIG. 4 illustrates a sectional view of an example of an arrangement of the one or more functional layers 124, the barrier layer 126, the barrier strip 128, and the body fabric layer 130 at the first side 120 and/or the second side 122 of the gusset 102.

As illustrated in FIG. 4, the one or more functional layers 124 include a body side surface 133 that faces the body of the user 101, and each of the one or more functional layers 124 includes opposing side edges 134. In FIG. 4, the functional layer 124A is a moisture-wicking layer and the functional layer 124B is an absorbent layer. However, as previously discussed, in other embodiments, a single functional layer 124 may be utilized and/or the functional layers 124 may be provided in various orders or arrangements as desired. As such, the particular functional layer 124A that defines the body side surface 133 should not be considered limiting.

The barrier layer 126 is provided on the garment side 114 of the one or more functional layers 124 and extends around the side edge(s) 134 of the one or more functional layers 124 such that the barrier layer 126 defines a side edge 144 having an external side 146. The barrier layer 126 additionally covers and/or extends over at least a portion of the body side surface 133 such that the barrier layer 126 has a topmost surface 136. In such embodiments, the barrier layer 126 may include a folded barrier layer portion 138 that is folded under the topmost surface 136. As mentioned, the barrier layer 126 is a laminate having the barrier side 140 and the fabric side 142, and in the embodiment illustrated in FIGS. 4 and 5, the fabric side 142 is between the one or more functional layers 124 and the barrier side 140. However, in other embodiments, the barrier layer 126 may be a single layer and/or provided in a different arrangement as desired. As a non-limiting example, in other embodiments, the barrier side 140 may be between the fabric side 142 and the one or more functional layers 124.

The barrier strip 128 may extend around the external side 146 of the side edge 144 of the barrier layer 126 such that the barrier strip 128 defines at least a portion of an outermost side edge 148 of the gusset 102 (and thus at least a portion of an outermost side edge of the reusable garment 100). In certain embodiments, the barrier strip 128 may extend over and/or or cover at least a portion of the body side surface 133 such that the barrier strip 128 has a topmost surface 150. In such embodiments, the barrier strip 128 may include a folded barrier strip portion 152 that is folded under the topmost surface 150 of the barrier strip 128. In some embodiments, the topmost surface 150 of the barrier strip 128 may be a topmost portion of the reusable garment 100 on the body side 112 of the reusable garment 100. In various embodiments, the topmost surface 150 may contact the skin 103 of the user 101 when the reusable garment 100, and such contact may provide leakage control at the first side 120 and/or the second side 122.

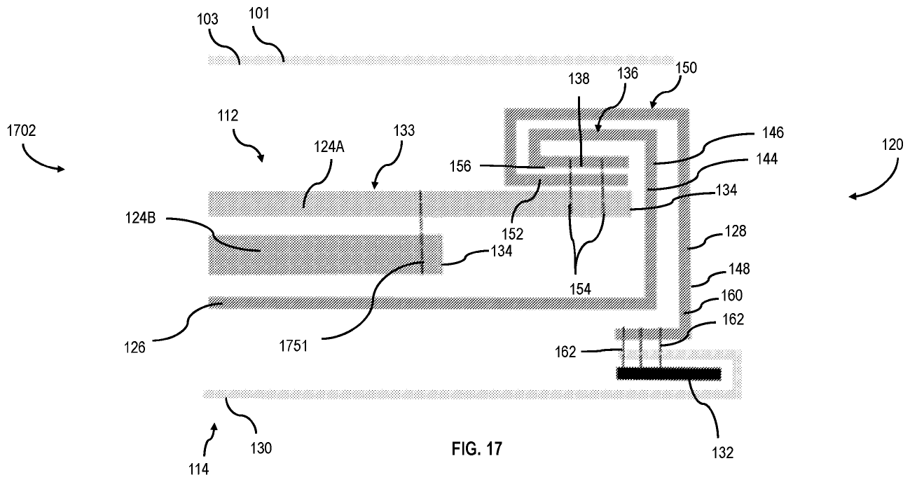

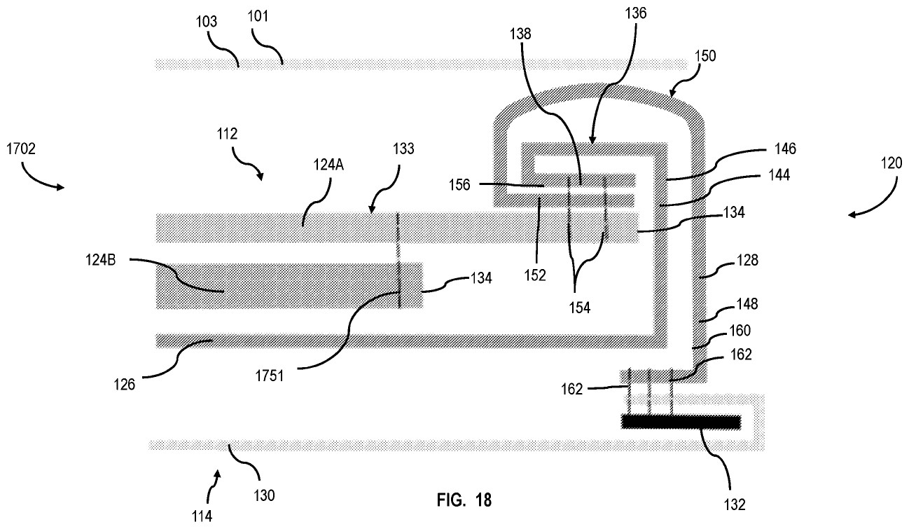

As illustrated in FIG. 4, for example, the topmost surface 150 of the barrier strip 128 may not be coupled to the topmost surface 136 of the barrier layer 126, thereby allowing for the topmost surface 150 to be “free-floating” or movable relative to the topmost surface 136. In such embodiments, the free-floating topmost surface 150 may allow for vertical movement and/or lateral movement. In some embodiments, the lack of coupling between the topmost surface 150 and the topmost surface 136 may allow for the topmost surface 150 to assume and/or appear as a tubular shape (e.g., a non-planar or curved shape) to the user (see, e.g., FIGS. 17 and 18). The topmost surface 150 not coupled to the topmost surface 136 thus may provide additional height to the barrier strip 128 without requiring additional materials, which may provide additional leakage protection and/or improvement in comfort and/or softness, flexibility, and/or fit of the gusset 102 against a user's skin.

Optionally, a width of the topmost surface 150 may be controlled (e.g., increased) to provide further leakage control at the first side 120 and/or the second side 122. As non-limiting examples, the width of the topmost surface 150 may be from about 2 mm to about 30 mm, such as about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, and/or about 30 mm. In one non-limiting example, the width of the topmost surface 150 may be from about 2 mm to about 15 mm. In another non-limiting example, the topmost surface 150 may be from about 25 mm to about 30 mm. In other embodiments, the topmost surface 150 may be less than about 2 mm and/or greater than about 30 mm as desired. In other embodiments the topmost surface 150 may have other widths as desired.

In some embodiments, the topmost surface 150 may have a varying width along a length of the front side 116, the back side 118, the first side 120, and/or the second side 122. As a non-limiting example, portions of the topmost surface 150 adjacent a waist area of the reusable garment 100 may be narrower than portions of the topmost surface 150 in a middle of the reusable garment 100. As another non-limiting example, the width of the topmost surface 150 of the front side 116 and/or the back side 118 may be different from the width of the topmost surface 150 of the first side 120 and/or the second side 122.

In some embodiments, the topmost surface 150 optionally may be controlled to be extend across a certain percentage of the width of the reusable garment 100 in a crotch area. As non-limiting example, the topmost surface 150 may be from about 1% to about 50% of the width of the reusable garment in the crotch area, such as less than about 45% of the width, such as less than about 40% of the width, such as less than about 35% of the width, such as less than about 30%, such as less than about 25% of the width, such as about 2% of the width, about 3% of the width, about 4% of the width, about 5% of the width, about 6% of the width, about 7% of the width, about 8% of the width, about 9% of the width, about 10% of the width, about 11% of the width, about 12% of the width, about 13% of the width, about 14% of the width, about 15% of the width, about 16% of the width, about 17% of the width, about 18% of the width, about 19% of the width, about 20% of the width, about 21% of the width, about 22% of the width, about 23% of the width, about 24% of the width, and/or about 25% of the width. Controlling the width of the topmost surface 150 relative to the width of the crotch area may further improve leakage control as well as comfort and fit for the wearer.

As mentioned, in the embodiment illustrated in FIG. 4, the barrier strip 128 is a laminate having the barrier side 158 and the fabric side 160. In this embodiment, the fabric side 160 may be a material similar to those suitable for the fabric side 142 of the barrier layer 126, and the barrier side 158 may be a material similar to those suitable for the barrier side 140 of the barrier layer 126, although it need not be in other embodiments. As illustrated in FIG. 4, the barrier layer 126 and the barrier strip 128 may be arranged relative to each other such that similar sides face and/or contact each other (e.g., the barrier side 158 faces and/or contacts the barrier side 140 as illustrated in FIG. 4). However, in other embodiments, the barrier layer 126 and the barrier strip 128 may be provided in various arrangements or configurations as desired.

In various embodiments, the barrier strip 128 may be coupled to the barrier layer 126 at a location above the body side surface 133 of the at least one functional layer 124. In certain embodiments, an adhesive-free and/or bonding-free coupling, such as but not limited to one or more first stitches 154, may couple the barrier layer 126 to the barrier strip 128. In some embodiments, the folded barrier strip portion 152 may be provided on a garment-facing side 156 of the folded barrier layer portion 138 of the barrier layer 128, and the one or more first stitches 154 may couple the folded barrier strip portion 152 to the folded barrier layer portion 138. The one or more first stitches 154 (or other suitable adhesive-free or bonding-free couplings) may further couple the folded barrier layer portion 138 and the folded barrier strip portion 152 to the one or more functional layers 124 without penetrating the barrier layer 128 on the garment side 114 of the one or more functional layers 124. Such a coupling may eliminate stitch holes in the barrier layer 126 on the garment side 114 of the one or more functional layers 124, thereby providing improved leakage control towards the garment side 114. Such a coupling may also provide two barriers (e.g., the barrier layer 126 and the barrier strip 128) around the side edge(s) 134 without stitching, thereby improving leakage control out the side of the reusable garment 100 and non-stitched barriers to liquid insults on the first side 120 and/or the second side 122. The adhesive-free or bonding-free coupling may further provide improved fit and comfort of the coupled materials to the user 101 when worn.

As illustrated in FIG. 4, the body fabric layer 130 is indirectly coupled to the barrier layer 126 via the barrier strip 128. In these embodiments, the barrier strip 128 is coupled to the body fabric layer 130 (and optionally the leg elastic 132) on the garment side 114 of the one or more functional layers 124. Coupling the barrier strip 128 to the body fabric layer 130 on the garment side 114 may provide improved leakage control as the location of the coupling is offset away from the coupling of the barrier strip 128 to the barrier layer 126 (thereby providing a greater distance of travel should the coupling of the barrier strip 128 to the barrier layer 126 be compromised). Such a coupling also offsets the coupling location away from the skin 103 of the user 101 with worn, thereby improving the durability of the coupling.

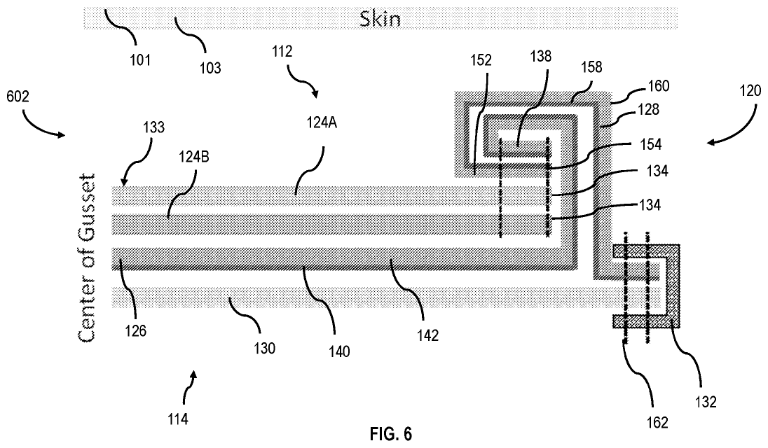

When the leg elastic 132 is included, the leg elastic 132 may be coupled to the body fabric layer 130 and the barrier layer 126. The leg elastic 132 may be provided at an internal location as illustrated in FIG. 4 (e.g., the body fabric layer 130 may enclose the leg elastic 132). In other embodiments, and as illustrated in FIG. 6, the leg elastic 132 may define at least a portion of the outermost side edge of the reusable garment.

In certain embodiments, an adhesive-free and/or bonding-free coupling, such as but not limited to one or more second stitches 162, may couple the barrier strip 128 to the body fabric layer 130 and optionally to the leg elastic 132. The adhesive-free and/or bonding-free coupling may maintain functionality of the leg elastic 132 (if included) and may provide an improved comfort and fit to the user 101.

FIG. 5 illustrates a sectional view of an optional example of an arrangement of the one or more functional layers 124, the barrier layer 126, and a second barrier strip 129, and the body fabric layer 130 at the front side 116 and/or the back side 118 of the gusset 102. However, in other embodiments, the one or more functional layers 124, the barrier layer 126, the barrier strip 128, and the body fabric layer 130 at the front side 116 and/or the back side 118 of the gusset 102 may have other configurations or arrangements as desired at the front side 116 and/or the back side 118. As a non-limiting example, in certain embodiments, the front side 116 and/or the back side 118 may have a construction or arrangement similar to that of the first side 120 and/or the second side 122 of the gusset 102. As a non-limiting example, similar to the first side 120 and/or the second side 122, the front side 116 and/or the back side 118 of the gusset 102 may include one or more functional layers 124, the barrier layer 126, the barrier strip 128, and the body fabric layer 130 at the first side 120 and/or the second side 122 of the gusset 102 as illustrated in FIG. 4.

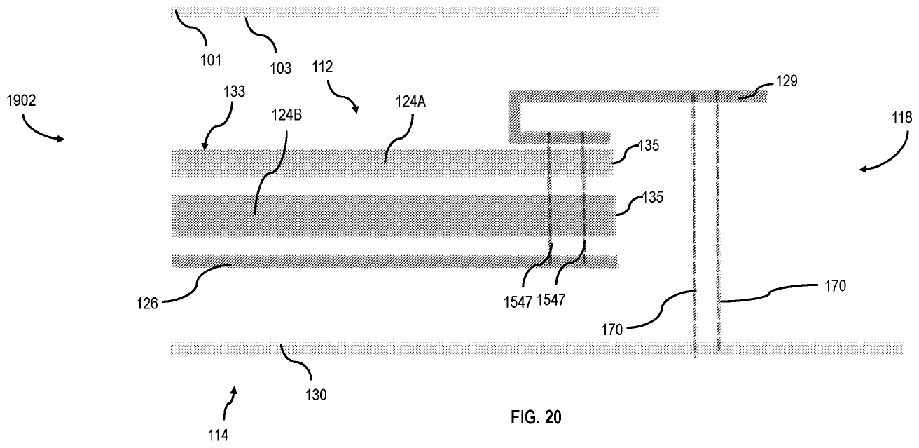

As illustrated in FIG. 5, the barrier layer 126 includes a front edge 166, and unlike at the first side 120 and/or the second side 122, the barrier layer 126 does not extend around a front (or back) edge 135 of the one or more functional layers 124. In certain embodiments, the second barrier strip 129 is substantially the same material as the barrier strip 128; however, it need not be in other embodiments. In the example illustrated in FIG. 5, the second barrier strip 129 includes a front edge 159 and is indirectly coupled to the barrier layer 126 using a sealing element 164, such as but not limited to a sealing tape. In this embodiment, the sealing element 164 may extend around the front edge(s) 135 of the one or more functional layers 124, the front edge 166 of the barrier layer 126, and the front edge 159 of the second barrier strip 129. Optionally, an adhesive layer 131, such as but not limited to a glue layer or other suitable adhesive, may be included to facilitate attachment of the sealing element 164 to the other layers of the reusable garment 100. However, in other embodiments, the adhesive layer 131 may be omitted. The arrangement with at least the sealing element 164 and optionally the adhesive layer 131 may enclose front edges 135, 159, 166 and provide a liquid impenetrable barrier seal. In other embodiments, the sealing element 164 and/or adhesive layer 131 may be omitted (see, e.g., FIGS. 20 and 21). As illustrated third stitches 170 or other suitable couplings may couple the body fabric layer 130 to the second barrier strip 129 and such that the body fabric layer 130 is indirectly coupled to the barrier layer 126.

FIG. 6 illustrates another example of a gusset 602 according to embodiments. The gusset 602 is similar to the gusset 102 and includes the one or more functional layers 124, the barrier layer 126, the barrier strip 128, the body fabric layer 130, and the leg elastic 132. Compared to the gusset 102, the leg elastic 132 of the gusset 602 forms at least a portion of the outermost side edge of a reusable garment incorporating the gusset 602.

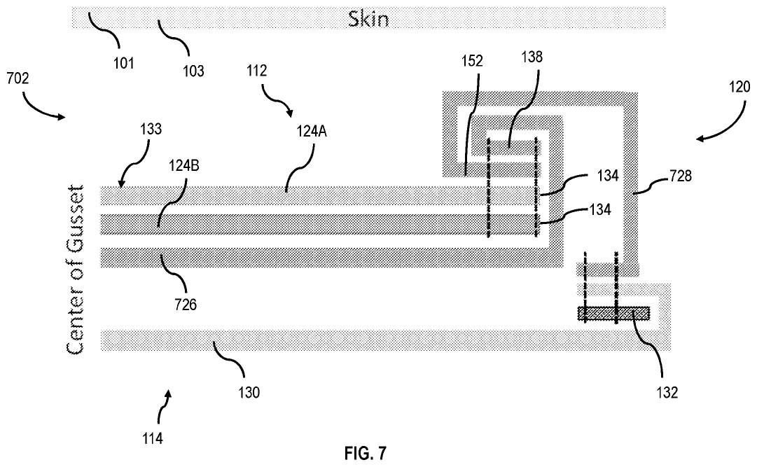

FIG. 7 illustrates another example of a gusset 702 according to embodiments. The gusset 702 is similar to the gusset 102 and includes that the one or more functional layers 124, a barrier layer 726, a barrier strip 728, the body fabric layer 130, and the leg elastic 132. Compared to the laminate constructions of the barrier layer 126 and the barrier strip 128 of the gusset 102, the barrier layer 726 and the barrier strip 728 of the gusset 702 are each single layers of material.

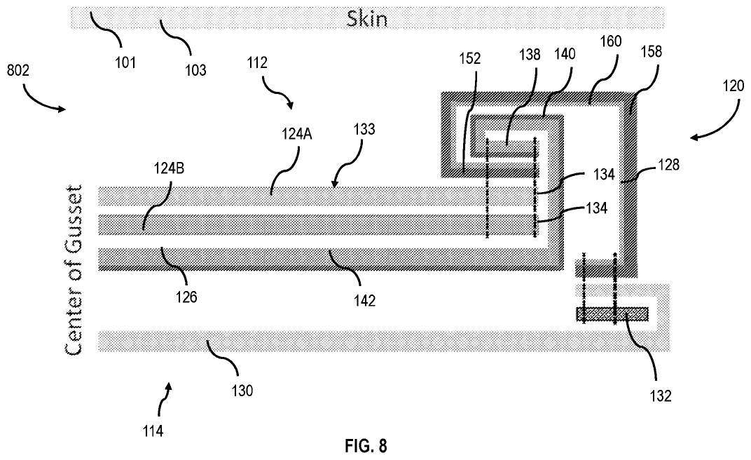

FIG. 8 illustrates another example of a gusset 802 according to embodiments. The gusset 802 is similar to the gusset 102 and includes the one or more functional layers 124, the barrier layer 126, the barrier strip 128, the body fabric layer 130, and the leg elastic 132. Compared to the gusset 102, the barrier layer 126 and the barrier strip 128 of the gusset 802 are arranged such that the barrier side 140 of the barrier layer 126 faces and/or is in contact with the fabric side 160 of the barrier strip 128.

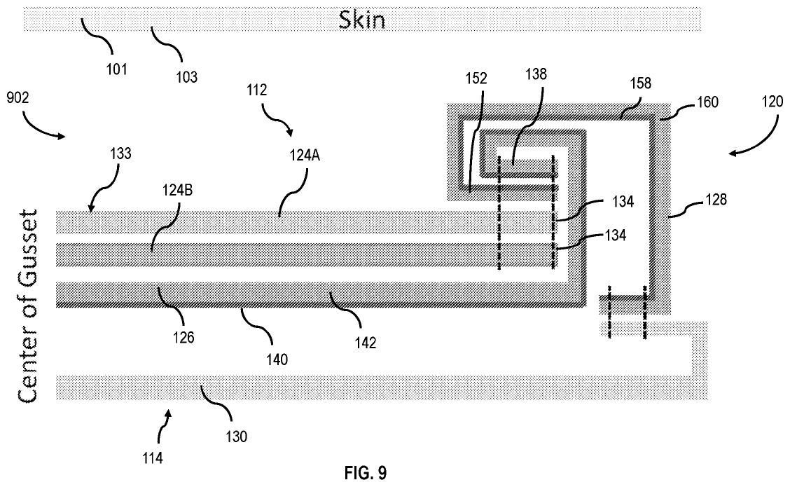

FIG. 9 illustrates another example of a gusset 902 according to embodiments. The gusset 902 is similar to the gusset 102 and includes the one or more functional layers 124, the barrier layer 126, the barrier strip 128, and the body fabric layer 130. Compared to the gusset 102, the gusset 902 omits the leg elastic 132.

FIG. 10 illustrates another example of a gusset 1002 according to embodiments. The gusset 1002 is similar to the gusset 102 and includes the one or more functional layers 124, the barrier layer 126, the barrier strip 128, the body fabric layer 130, and the leg elastic 132. Compared to the gusset 102, the gusset 1002 includes a single functional layer 124. In this example, the single functional layer may be an absorbent material and/or a moisture-wicking material.

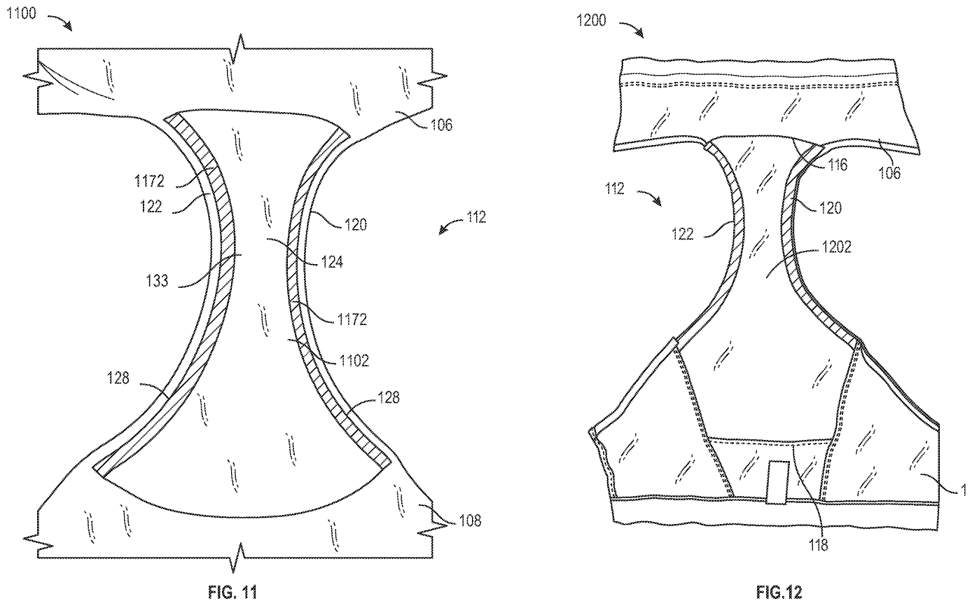

FIG. 11 illustrates another example of a reusable garment 1100 with a gusset 1102 according to embodiments. The reusable garment 1100 and the gusset 1102 are similar to the reusable garment 100 and the gusset 102 except that the gusset 1102 further includes a visual cue or indicator 1172 along the first side 120 and/or the second side 122 with a visual characteristic different from one or more other portions of the reusable garment 1100. In one non-limiting example, the visual characteristic may be different from another portion of the reusable garment 1100 and/or the gusset 1102 on the body side 112.

The visual indicator 1172 may be various types of visual indicators as desired, such as but not limited to colors, patterns, shapes, surface roughness, surface smoothness, types of materials (e.g., different types of fabrics), an absence of stitching or threading, a continuous surface, features providing different light reflectance and/or sheen, combinations thereof, and/or other visual indicators as desired. The visual indicators 1172 may be integral with one or more portions of the gusset 1102 and/or may be separate components attached and/or otherwise coupled to the gusset 1102. In embodiments with the visual indicator 1172, the visual indicator 1172 may be provided on and/or as portions of various components of the gusset 1102, such as but not limited to the barrier strip 128, the second barrier strip 129, leg elastic 132 (e.g., when leg elastic 132 is folded over), combinations thereof, and/or other portions as desired. In one non-limiting example, the visual indicator 1172 may at least a portion of the topmost surface 150 of the barrier strip 128, and the topmost surface 150 of the barrier strip 128 includes different colors, patterns, and/or shapes compared to the body side surface 133 of the functional layer 124. As another non-limiting example, the fabric side 160 and/or the barrier side 158 of the barrier strip 128 may provide different light reflectance than the body side surface 133 of the functional layer 124.

When included, the visual indicator 1172 may provide an improved visual appearance to the reusable garment 1100 while also providing a reference to facilitate positioning and/or use with the user. Additionally, or alternatively, the visual indicators 1172 may convey information to a wearer. As a non-limiting example, the fabric side 160 and/or the barrier side 158 of the barrier strip 128 may provide different light reflectance than the body side surface 133 of the functional layer 124 indicating the non-absorbing nature of the bag-out construction and improved barrier performance. As a non-limiting example, both the topmost surface 150 of the barrier strip 128 and the body side surface 133 of the functional layer 124 may be dyed black, but the difference in materials used for these surfaces 150, 133 (e.g., thereby providing different sheen or light reflectance) may create different user perceptions, thereby facilitating the conveyance of the leakage performance of the barrier strip 128 to the user.

Optionally, the visual indicators 1172 may convey information by changing a characteristic of the visual indicators 1172. As a non-limiting example, the visual indicators 1172 may change color, pattern, etc. to indicate a fullness or saturation to a wearer.

Moreover, while illustrated on the first side 120 and the second side 122, in other embodiments, the visual indicator 1172 may be on the front side 116 and/or the back side 118 as desired, and the visual indicator need not be on the first side 120 and/or the second side 122. When a plurality of sides 116, 118, 120, 122 include visual indicators 1172, they may be similar visual indicators (e.g., all having a same logo, color, pattern, and/or other characteristic), although in other embodiments, a characteristic of one visual indicator 1172 may be different from a characteristic of another visual indicator 1172. As a non-limiting example, visual indicators 1172 along the sides 116, 118 (e.g., defined by the barrier strips 128) may be a gold color, and visual indicators 1172 along sides 120, 122 (e.g., defined by the second barrier strips 129) may have a green color. Other variations of characteristics may be utilized as desired.

FIGS. 12-14 illustrate another example of a reusable garment 1200 with a gusset 1202 according to embodiments. The reusable garment 1200 and the gusset 1202 are similar to the reusable garment 100 and the gusset 102 except that the gusset 1202 has a different shape compared to the gusset 102. As best illustrated in FIG. 14, the gusset 1202 additionally include a linear portion 1274 on the first side 120 and a linear portion 1276 on the second side 122.

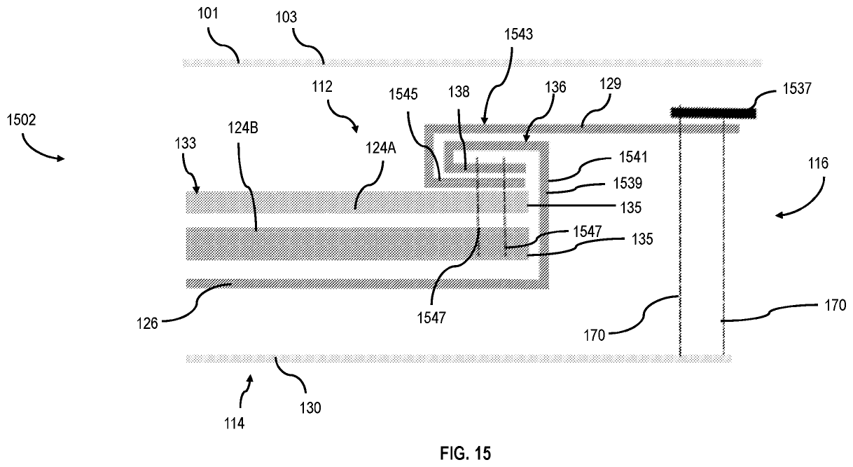

FIG. 15 illustrates another gusset 1502 according to embodiments. Similar to the gusset 102 at the front side 116 and/or the back side 118 as illustrated in FIG. 5, the gusset 1502 at the front side 116 and/or the back side 118 may include third stitches 170 or other suitable couplings may couple the body fabric layer 130 to the second barrier strip 129 and such that the body fabric layer 130 is indirectly coupled to the barrier layer 126. Optionally, the gusset 1502 includes a spreader elastic 1505, and the spreader elastic 1537 may be coupled to the gusset 1502 via the third stitches 170 or outer suitable couplings.

Compared to the gusset 102 at the front side 116 and/or the back side 118 of the gusset 1502, the gusset 1502 omits the sealing element 164. In this embodiment, the barrier layer 126 extends around the front (or back) edge(s) 135 of the one or more functional layers 124 such that the barrier layer 126 defines a front (or back) edge 1539 having an external side 1541. The barrier layer 126 additionally covers and/or extends over at least a portion of the body side surface 133 such that the barrier layer 126 has the topmost surface 136 and optionally with the folded barrier layer portion 138.

Similar to the barrier strip 128 illustrated in FIG. 4, as illustrated in FIG. 15, the second barrier strip 129 extend over and/or or cover at least a portion of the body side surface 133 such that the second barrier strip 129 has a topmost surface 1543, which may be similar to the topmost surface 150. In certain embodiments, similar to the topmost surface 150, the topmost surface 1543 may be free-floating. In various embodiments, similar to the barrier strip 128, the second barrier strip 129 may include a second folded barrier strip portion 1545 that is folded under the topmost surface 1543 of the second barrier strip 129. In some embodiments, similar to the topmost surface 150, the topmost surface 1543 of the second barrier strip 129 may be a topmost portion of the reusable garment on the body side 112 of the reusable garment 100. In various embodiments, the topmost surface 1543 may contact the skin 103 of the user 101 when the reusable garment is worn, and such contact may provide leakage control at the front side 116 and/or the back side 118.

In certain embodiments, an adhesive-free and/or bonding-free coupling, such as but not limited to one or more fourth stitches 1547, may couple the barrier layer 126 to the second barrier strip 129. In some embodiments, the one or more fourth stitches 1547 may couple the second folded barrier strip portion 1545 to the folded barrier layer portion 138. The one or more fourth stitches 1547 (or other suitable adhesive-free or bonding-free couplings) may further couple the folded barrier layer portion 138 and the second folded barrier strip portion 1545 to the one or more functional layers 124 without penetrating a barrier layer on the garment side 114 of the one or more functional layers 124.

FIG. 16 illustrates another gusset 1602 according to embodiments. The gusset 1602 is similar to the gusset 1502 except that the functional layers 124A-B are non-homogeneous, and the edges 135 are not aligned. In this embodiment, the gusset 1602 optionally includes one or more fifth stitches 1649 coupling the functional layers 124A-B.

FIGS. 17 and 18 illustrate another gusset 1702. The gusset 1702 is similar to the gusset 102 as illustrated in FIG. 4 except that the functional layers 124A-B are non-homogeneous, and the edges 134 are not aligned. In this embodiment, the gusset 1702 optionally includes one or more sixth stitches 1751 coupling the functional layers 124A-B. FIG. 18 further illustrates vertical movement of the free-floating topmost surface 150 such that the topmost surface 150 of the barrier strip 128 may achieve a tubular, curved, and/or otherwise non-planar shape. As mentioned, the free-floating topmost surface 150 may be laterally movable in addition to being vertically movable relative to the topmost surface 136.

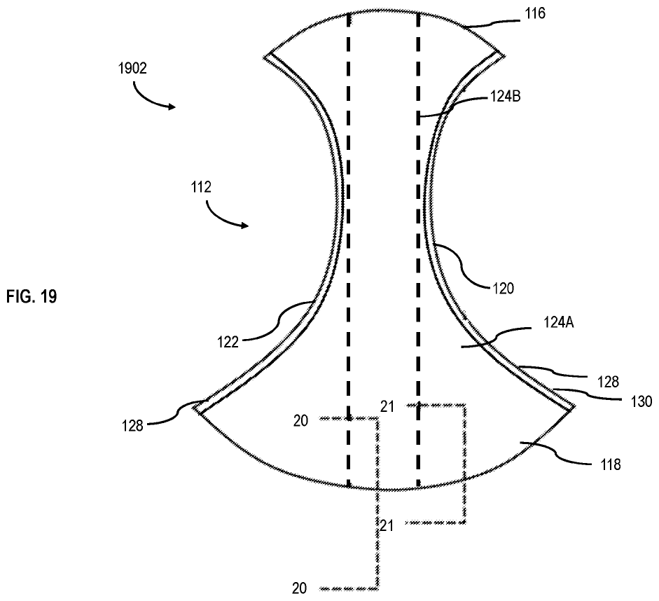

FIGS. 19-21 illustrate another example of a gusset 1902 according to embodiments. The gusset 1902 is similar to the gusset 102 except that the functional layers 124A-B of the gusset 1902 are non-homogeneous. As non-limiting examples, at some portions of the front side 116 and/or the back side 118 (and/or the sides 120, 122), both functional layers 124A-B may be present as illustrated in FIG. 20, while at other portions of the front side 116 and/or the back side 118 (and/or the sides 120, 122), only one functional layer may be present (e.g., functional layer 124A as illustrated in FIG. 21). In FIG. 19, the positioning of the functional layer 124B under the functional layer 124A is illustrated in dashed lines. As previously mentioned, various other combinations of one or more functional layers may be utilized with gussets described herein as desired.

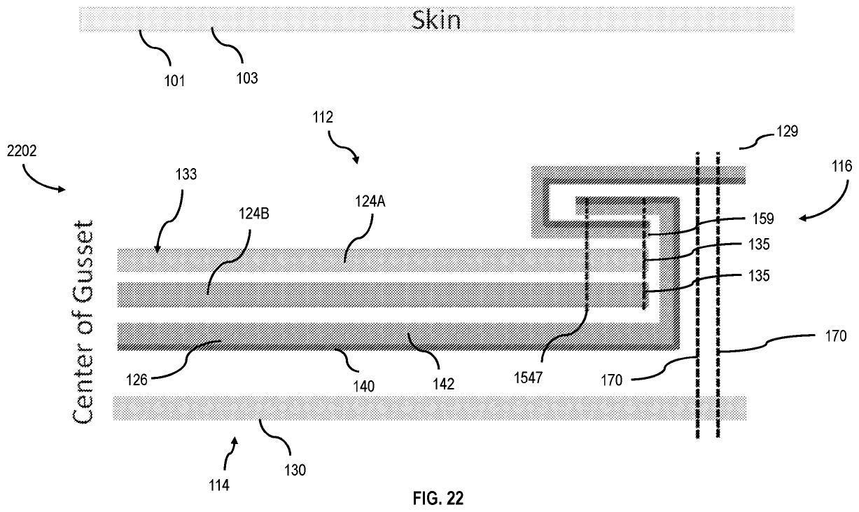

FIG. 22 illustrates another example of a gusset 2202 according to embodiments. The gusset 2202 is similar to the gusset 102 except that at the front side 116 and/or the back side 118, the gusset 2202 omits the sealing element 164, and instead the barrier layer 126 extends up and around the front edges 135 of the functional layers 124A-B. Optionally, the barrier layer 126 may extend up and around the front edges 135 in a manner similar to that at the side edges (e.g., as illustrated in FIG. 4, for example).

As mentioned, the gussets described herein and reusable garments incorporating such gussets may have improved leakage control compared to traditional approaches. The gussets described herein may include two layers that wrap around side edges of one or more functional layers of the gusset that form barriers to liquid insults at side edges of the gusset (and thus side edges of the reusable garment). The gussets described herein have improved coupling locations where the barrier layer is coupled to the barrier strip and where the barrier strip is coupled to the body fabric layer, which may further minimize and/or prevent leakage through stitching holes. In certain embodiments, the gussets described herein may include couplings that are adhesive-free and/or bonding-free, which may maintain functionality of leg elastic and/or otherwise provide improved comfort and fit to a user. Detachment of the functional layer(s) from the body fabric (e.g., they are indirectly coupled), may further minimize and/or prevent leakage compared to traditional approaches. In certain embodiments, the gussets described herein may provide a barrier to liquid insults at least at side edges of the gusset. Additionally, or alternatively, the gussets described herein may provide a barrier to liquid insults along all edges of the gusset (e.g., at front and back sides as well as side edges), which may further provide an improved garment compared to traditional approaches. In some embodiments, providing a barrier along all edges of the gusset may improve modularity of the reusable garment and allow for the reusable garment to be utilized in a variety of use scenarios, and the reusable garment need not be a product with a specialized end use. Various other benefits and advantages may be realized with the systems and methods provided herein, and the aforementioned advantages should not be considered limiting.

A collection of exemplary embodiments is provided below, including at least some explicitly enumerated as “Embodiments” providing additional description of a variety of example embodiments in accordance with the concepts described herein. These embodiments are not meant to be mutually exclusive, exhaustive, or restrictive; and the disclosure not limited to these example embodiments but rather encompasses all possible modifications and variations within the scope of the issued claims and their equivalents.

Embodiment 1. A reusable garment comprising: a functional layer comprising a body side surface on a body side of the reusable garment; a barrier layer; a barrier strip coupled to the barrier layer and extending around an external side of a first side edge of the barrier layer, the barrier strip defining at least a portion of an outermost side edge of the reusable garment; and a body fabric layer.

Embodiment 2. The reusable garment of any of the preceding or subsequent embodiments or combination of embodiments, wherein the functional layer comprises at least one of a wicking layer or an absorbent layer.

Embodiment 3. The reusable garment of any of the preceding or subsequent embodiments or combination of embodiments, wherein the functional layer is a wicking and absorbent layer.

Embodiment 4. The reusable garment of any of the preceding or subsequent embodiments or combination of embodiments, wherein the functional layer comprises a wicking layer and an absorbent layer, and wherein the absorbent layer is disposed between the wicking layer and the barrier layer.

Embodiment 5. The reusable garment of any of the preceding or subsequent embodiments or combination of embodiments, wherein the barrier layer and the barrier strip form two non-stitched barriers to liquid insults on a side edge of the functional layer.

Embodiment 6. The reusable garment of any of the preceding or subsequent embodiments or combination of embodiments, wherein the barrier layer comprises a laminate comprising a barrier side and a fabric side.

Embodiment 7. The reusable garment of any of the preceding or subsequent embodiments or combination of embodiments, wherein the barrier strip comprises a laminate comprising a barrier side and a fabric side.

Embodiment 8. The reusable garment of any of the preceding or subsequent embodiments or combination of embodiments, further comprising a leg elastic, wherein the leg elastic is coupled to the body fabric and the barrier strip.

Embodiment 9. The reusable garment of any of the preceding or subsequent embodiments or combination of embodiments, wherein the leg elastic defines at least a portion of the outermost side edge of the reusable garment.

Embodiment 10. The reusable garment of any of the preceding or subsequent embodiments or combination of embodiments, wherein the barrier strip defines a topmost portion of the reusable garment on the body side of the reusable garment.

Embodiment 11. The reusable garment of any of the preceding or subsequent embodiments or combination of embodiments, wherein the functional layer and the barrier layer each comprise a front edge, wherein the barrier strip is a first barrier strip, and wherein the reusable garment further comprises: a second barrier strip coupled to the functional layer proximate to the front edge of the functional layer, the second barrier strip comprising a front edge; and a sealing tape extending around an external side of the front edge of the second barrier strip, the front edge of the barrier layer, and the front edge of the functional layer.

Embodiment 12. The reusable garment of any of the preceding or subsequent embodiments or combination of embodiments, wherein the second barrier strip overlaps the sealing tape and defines a topmost portion of the reusable garment on the body side of the reusable garment.

Embodiment 13. The reusable garment of any of the preceding or subsequent embodiments or combination of embodiments, wherein the second barrier strip comprises a folded barrier strip portion folded under a topmost surface of the second barrier strip and an unfolded barrier strip portion, wherein the sealing tape is disposed on a body side of the folded barrier strip portion.

Embodiment 14. The reusable garment of any of the preceding or subsequent embodiments or combination of embodiments, wherein a stitch couples the unfolded barrier strip portion to the body fabric.

Embodiment 15. A reusable garment comprising: a functional layer comprising a body side surface on a body side of the reusable garment; a barrier layer; a barrier strip coupled to the barrier layer; and a body fabric layer coupled to the barrier strip on a garment side of the functional layer.

Embodiment 16. The reusable garment of any of the preceding or subsequent embodiments or combination of embodiments, wherein the functional layer comprises at least one of a wicking layer or an absorbent layer.

Embodiment 17. The reusable garment of any of the preceding or subsequent embodiments or combination of embodiments, wherein the functional layer is a wicking and absorbent layer.

Embodiment 18. The reusable garment of any of the preceding or subsequent embodiments or combination of embodiments, wherein the functional layer comprises a wicking layer and an absorbent layer, and wherein the absorbent layer is disposed between the wicking layer and the barrier layer.

Embodiment 19. The reusable garment of any of the preceding or subsequent embodiments or combination of embodiments, wherein the barrier layer and the barrier strip form two non-stitched barriers to liquid insults on a side edge of the functional layer.

Embodiment 20. The reusable garment of any of the preceding or subsequent embodiments or combination of embodiments, wherein the barrier layer comprises a laminate comprising a barrier side and a fabric side.

Embodiment 21. The reusable garment of any of the preceding or subsequent embodiments or combination of embodiments, wherein the barrier strip comprises a laminate comprising a barrier side and a fabric side, wherein a portion of the barrier side of the barrier layer contacts a portion of the barrier side of the barrier strip.

Embodiment 22. The reusable garment of any of the preceding or subsequent embodiments or combination of embodiments, further comprising a leg elastic, wherein the leg elastic is coupled to the body fabric and the barrier strip.

Embodiment 23. The reusable garment of any of the preceding or subsequent embodiments or combination of embodiments, wherein an adhesive-free coupling couples the body fabric layer to the barrier strip.

Embodiment 24. The reusable garment of any of the preceding or subsequent embodiments or combination of embodiments, wherein the body fabric layer is coupled to the barrier strip on a garment side of the barrier layer.

Embodiment 25. A reusable garment comprising: a functional layer; a barrier layer, wherein the barrier layer comprises a folded barrier layer portion under a topmost surface of the barrier layer; a barrier strip including a folded barrier strip portion folded under a topmost surface of the barrier strip, wherein the folded barrier strip portion is disposed on a garment-facing side of the folded barrier layer portion of the barrier layer; and a body fabric layer, wherein a stitch couples the folded barrier layer portion to the folded barrier strip portion.

Embodiment 26. The reusable garment of any of the preceding or subsequent embodiments or combination of embodiments, wherein the stitch further couples the folded barrier strip portion to the functional layer.

Embodiment 27. The reusable garment of any of the preceding or subsequent embodiments or combination of embodiments, wherein the stitch is a first stitch, and wherein the reusable garment further comprises a second stitch coupling the barrier strip to the body fabric layer.

Embodiment 28. The reusable garment of any of the preceding or subsequent embodiments or combination of embodiments, further comprising a leg elastic, wherein the second stitch couples the leg elastic to the barrier strip and the body fabric layer.

Embodiment 29. The reusable garment of any of the preceding or subsequent embodiments or combination of embodiments, wherein the barrier strip defines at least a portion of an outermost side edge of the reusable garment.

Embodiment 30. The reusable garment of any of the preceding or subsequent embodiments or combination of embodiments, wherein the functional layer comprises at least one of a wicking layer or an absorbent layer.

Embodiment 31. The reusable garment of any of the preceding or subsequent embodiments or combination of embodiments, wherein the functional layer is a wicking and absorbent layer.

Embodiment 32. The reusable garment of any of the preceding or subsequent embodiments or combination of embodiments, wherein the functional layer comprises a wicking layer and an absorbent layer, wherein the absorbent layer is disposed between the wicking layer and the barrier layer.

Embodiment 33. The reusable garment of any of the preceding or subsequent embodiments or combination of embodiments, wherein the stitch further couples the wicking layer to the absorbent layer.

Embodiment 34. A reusable garment comprising: a functional layer comprising a body side surface on a body side of the reusable garment; a barrier layer extending around an edge of the functional layer, the barrier layer comprising a topmost surface and a folded barrier layer portion which is folded under the topmost surface of the barrier layer; a barrier strip coupled to the barrier layer, the barrier strip comprising a topmost surface and a folded barrier strip portion, wherein the folded barrier strip portion is folded under the topmost surface of the barrier strip and under the folded barrier layer portion; and a body fabric layer.

Embodiment 35. The reusable garment of any of the preceding or subsequent embodiments or combination of embodiments, wherein the edge of the functional layer is a front edge, a back edge, or a side edge.

Embodiment 36. The reusable garment of any of the preceding or subsequent embodiments or combination of embodiments, wherein the topmost surface of the barrier strip is decoupled from the topmost surface of the barrier layer such that the topmost surface of the barrier strip is free-floating relative to the topmost surface of the barrier layer.

Embodiment 37. The reusable garment of any of the preceding or subsequent embodiments or combination of embodiments, wherein the topmost surface of the barrier strip is a topmost portion of the reusable garment on a body side of the reusable garment.

Embodiment 38. A reusable garment comprising: a functional layer comprising a body side surface on a body side of the reusable garment; a barrier layer extending around an edge of the functional layer, the barrier layer comprising a topmost surface and a folded barrier layer portion which is folded under the topmost surface of the barrier layer; a barrier strip coupled to the barrier layer, the barrier strip comprising a topmost surface extending over the topmost surface of the barrier layer, wherein the topmost surface of the barrier strip is free-floating relative to the topmost surface of the barrier layer; and a body fabric layer.

Embodiment 39. The reusable garment of any of the preceding or subsequent embodiments or combination of embodiments, wherein the topmost surface of the barrier strip further comprises a visual indicator.

Embodiment 40. The reusable garment of any of the preceding or subsequent embodiments or combination of embodiments, wherein the topmost surface of the barrier strip is movable into non-planar shape relative to the topmost surface of the barrier layer.

Embodiment 41. A reusable garment comprising: a functional layer comprising a body side surface on a body side of the reusable garment; a barrier layer extending around an edge of the functional layer, the barrier layer comprising a topmost surface and a folded barrier layer portion which is folded under the topmost surface of the barrier layer; a barrier strip coupled to the barrier layer, the barrier strip comprising a topmost surface extending over the topmost surface of the barrier layer, wherein the topmost surface of the barrier strip further comprises a visual indicator with a visual characteristic different from another portion of the reusable garment on the body side; and a body fabric layer.

Embodiment 42. The reusable garment of any of the preceding or subsequent embodiments or combination of embodiments, wherein the visual characteristic comprises at least one of a color, a pattern, or a shape.

Embodiment 43. The reusable garment of any of the preceding or subsequent embodiments or combination of embodiments, wherein the visual characteristic comprises a light reflectance.

Embodiment 44. The reusable garment of any of the preceding or subsequent embodiments or combination of embodiments, wherein the visual characteristic comprises a surface roughness.

Embodiment 45. The reusable garment of any of the preceding or subsequent embodiments or combination of embodiments, wherein the visual characteristic is adjustable.

Embodiment 46. The reusable garment of any of the preceding or subsequent embodiments or combination of embodiments, wherein the visual characteristic comprises at least one of a continuous surface and/or an absence of stitching or threading.

The various aspects, embodiments, implementations or features of the described embodiments can be used separately or in any combination. It should be appreciated that the various elements of concepts from the figures may be combined without departing from the spirit or scope of the invention.

The use of the terms “a” and “an” and “the” and similar referents in the context of describing the invention (especially in the context of the following claims) are to be construed to cover both the singular and the plural, unless otherwise indicated herein or clearly contradicted by context. Directional references such as “up,” “down,” “top,” “bottom,” “left,” “right,” “front,” and “back,” among others, are intended to refer to the orientation as illustrated and described in the figure (or figures) to which the components and directions are referencing. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, or gradients thereof, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, the nearness of completion will be so as to have the same overall result as if absolute and total completion were obtained.

Preferred embodiments of this invention are described herein, including the best mode known to the inventors for carrying out the invention. The invention is susceptible to various modifications and alternative constructions, and certain shown exemplary embodiments thereof are shown in the drawings and have been described above in detail. Variations of those preferred embodiments, within the spirit of the present invention, may become apparent to those of ordinary skill in the art upon reading the foregoing description. The inventors expect skilled artisans to employ such variations as appropriate, and the inventors intend for the invention to be practiced otherwise than as specifically described herein. Accordingly, it should be understood that there is no intention to limit the invention to the specific form or forms disclosed, but on the contrary, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view of the above teachings.

That which is claimed:

1. A reusable garment comprising:
a functional layer comprising a body side surface on a body side of the reusable garment;
a barrier layer extending around an edge of the functional layer, the barrier layer comprising a topmost surface;
a barrier strip coupled to the barrier layer, the barrier strip comprising a topmost surface and a folded barrier strip portion, wherein the folded barrier strip portion is folded under the topmost surface of the barrier strip and under the folded barrier layer portion; and
a body fabric layer.

2. The reusable garment of claim 1, wherein the functional layer comprises at least one of a wicking layer, an absorbent layer, or a wicking and absorbent layer

3. The reusable garment of claim 1, wherein the functional layer comprises a wicking layer and an absorbent layer, and wherein the absorbent layer is disposed between the wicking layer and the barrier layer.

4. The reusable garment of claim 1, wherein the edge of the functional layer is a front edge, a back edge, or a side edge.

5. The reusable garment of claim 1, wherein the barrier layer comprises a laminate comprising a barrier side and a fabric side, and/or wherein the barrier strip comprises a laminate comprising a barrier side and a fabric side.

6. The reusable garment of claim 1, wherein the topmost surface of the barrier strip is decoupled from the topmost surface of the barrier layer such that the topmost surface of the barrier strip is free-floating relative to the topmost surface of the barrier layer.

7. The reusable garment of claim 1, wherein the topmost surface of the barrier strip is a topmost portion of the reusable garment on a body side of the reusable garment.

8. A reusable garment comprising:
a functional layer comprising a body side surface on a body side of the reusable garment;
a barrier layer extending around an edge of the functional layer, the barrier layer comprising a topmost surface and a folded barrier layer portion which is folded under the topmost surface of the barrier layer;
a barrier strip coupled to the barrier layer, the barrier strip comprising a topmost surface extending over the topmost surface of the barrier layer, wherein the topmost surface of the barrier strip is free-floating relative to the topmost surface of the barrier layer; and
a body fabric layer.

9. The reusable garment of claim 8, wherein the functional layer comprises at least one of a wicking layer, an absorbent layer, or a wicking and absorbent layer

10. The reusable garment of claim 8, wherein the functional layer comprises a wicking layer and an absorbent layer, and wherein the absorbent layer is disposed between the wicking layer and the barrier layer.

11. The reusable garment of claim 8, wherein the edge of the functional layer is a front edge, a back edge, or a side edge.

12. The reusable garment of claim 8, wherein the barrier layer comprises a laminate comprising a barrier side and a fabric side, and/or wherein the barrier strip comprises a laminate comprising a barrier side and a fabric side.

13. The reusable garment of claim 8, wherein the topmost surface of the barrier strip further comprises a visual indicator.

14. The reusable garment of claim 8, wherein the topmost surface of the barrier strip is movable into non-planar shape relative to the topmost surface of the barrier layer.

15. A reusable garment comprising:
a functional layer comprising a body side surface on a body side of the reusable garment;
a barrier layer extending around an edge of the functional layer, the barrier layer comprising a topmost surface and a folded barrier layer portion which is folded under the topmost surface of the barrier layer;
a barrier strip coupled to the barrier layer, the barrier strip comprising a topmost surface extending over the topmost surface of the barrier layer, wherein the topmost surface of the barrier strip further comprises a visual indicator with a visual characteristic different from another portion of the reusable garment on the body side; and
a body fabric layer.

16. The reusable garment of claim 15, wherein the visual characteristic comprises at least one of a color, a pattern, or a shape.

17. The reusable garment of claim 15, wherein the visual characteristic comprises a light reflectance.

18. The reusable garment of claim 15, wherein the visual characteristic comprises a surface roughness.

19. The reusable garment of claim 15, wherein the visual characteristic is adjustable.

20. The reusable garment of claim 15, wherein the visual characteristic comprises at least one of a continuous surface and/or an absence of stitching or threading.
